(19) United States
(12) Patent Application Publication
(10) Pub. No.: US 2017/0216071 A1
(43) Pub. Date: Aug. 3, 2017

(54) Ankle-foot varus deformity alignment device

(71) Applicant: Wade Bader, Lurtz, FL (US)

(72) Inventor: Wade Bader, Lurtz, FL (US)

(21) Appl. No.: 14/999,559

(22) Filed: May 24, 2016

Related U.S. Application Data
(60) Provisional application No. 62/388,615, filed on Feb. 2, 2016.

(51) Int. Cl. A61F 5/01

(52) U.S. Cl. CPC A61F 5/0111; A61F 5/0113

(57) ABSTRACT

An ankle-foot varus deformity alignment device to improve leg, foot and ankle alignment of a person suffering from an ankle-foot varus deformity comprising an elongated spiral configured interconnecting member extending between an upper calf cuff including a medial region adapted to engage the medial side of the upper portion of the calf and a lower foot support including a lateral ankle cuff adapted to engage the lateral side of the ankle and a medial foot restraint adapted to engage in medial side of the foot to improve leg, ankle and foot alignment.

### CROSS-REFERENCE

This is a utility application of co-pending provisional application Ser. No. 62/388,615, filed Feb. 2, 2016.

### BACKGROUND OF THE INVENTION

Field of the Invention

An ankle-foot varus deformity alignment device.

Description of the Prior Art

Ankle-foot varus deformities are some what common to various degrees of severity.

In extremely severe cases surgery is dictated. However, in less severe cases orthotic corrective devices may be sufficient to alleviate or lessen impact of the deformity.

WO 2005/034819 discloses an orthosis for the lower leg comprising has a unitary body with a generally spiral shaped lower leg portion and a foot plate portion. The orthosis is configured to allow simultaneous control of the knee and ankle-foot complex during locomotion.

U.S. Pat. No. 7,018,352 relates to a modular ankle-foot orthosis comprising a lower leg and ankle support and a plantar foot support. The two supports may be attached in various ways, such as a bottom heel clip extending from the foot support component, which secures through a passage in the heel of the lower leg and ankle support component. A tongue extends from the foot support clip, to capture the material at the edge of the lower leg and ankle support passage between the tongue and the foot support plate.

U.S. Pat. No. 5,897,515 shows an ankle-foot orthosis comprising a frame of thin flexible material extending over the front of the lower leg, anterior of the lateral ankle and beneath the sole of the foot and a supporting portion of rigid material extending over a narrow part of the front of the lower leg, anterior of the lateral ankle and beneath the part of the sole of the foot.

U.S. Pat. No. 6,887,213 describes an ankle-foot orthosis comprising a strut extending over the front of the lower leg and anterior of the lateral ankle, a foot plate extending beneath the sole of the foot and a fastening means for the fastening the orthosis to the leg. The strut comprises a bifurcation zone and two strut branches arranged to extend on the outside of the lower leg on each side of the tibia.

U.S. Pat. No. 5,458,565 teaches an osteoarthritic knee brace having flexible upper and lower arm members rotatably connected to each other by a rotary hinge assembly. A selectively inflatable or deflatable fluid-containing pad is positionable between the hinge assembly and a side of the knee joint. In operation, the brace is mounted on the leg with the pad engaging both the hinge assembly and the side of the knee joint, the hinge assembly aligned with the knee joint, and the upper and lower arm members aligned with the upper and lower legs, respectively. The upper and lower arm members are stressed away from the knee joint thereby applying a restoring force to the knee joint across the hinge assembly and the pad that effectively treats the osteoarthritic knee joint

U.S. Pat. No. 5,887,591 describes a restraint and method for stretching the plantar fascia comprising a foot plate in combination with a lifting member to elevate the phalanges and to reduce contracture of the MTP joints.

U.S. Pat. No. 6,146,344 relates to a lower limb orthotic brace to control the movement of the lower limb of a patient comprising a contoured orthotic contact shell and a integral load-bearing frame. The contoured orthotic contact shell comprises at least one layer of fabric impregnated with a hardened structural resin forming an inner orthotic contact surface conforming to the outer surface of the lower limb of the patient. The integral load-bearing frame comprises at least one reinforcing member extending along the contoured orthotic contact shell including a plurality of bundled continuous composite fibers woven in a multidirectional tubular braid impregnated with a hardened structural resin with at least one side of the braid in contact with the contoured orthotic contact shell along the entire length of the braid such that the hardened resins impregnating the fabric of the contoured orthotic contact shell combine with the resins impregnating the braid thereby forming a continuous integral structural matrix encapsulating both the braid and the contoured orthotic contact shell.

U.S. Pat. No. 6,689,081 shows a therapeutic ankle and foot orthosis or brace comprising a lower shell and an upper lateral shell movably coupled together by a pivot so that the shells accommodate the contour, size and shape of the user's foot and ankle. Cushions are included on the shells to provide comfort and straps with hook and pile fasteners are employed to detachably retain the combined shells on the ankle and front of the user.

U.S. Pat. No. 4,446,856 describes an orthotic device comprising a boot for immobilizing the ankle and foot against relative movement and holds the ankle and foot in a neutral position of maximal joint congruency while providing a sole having a platform for causing the foot to follow a heel to toe rocking movement such that the leg of the user is flexed at the knee and a pivoting motion is induced at the hip joint during walking.

US 2011/0319799 teaches an ankle foot orthosis to correct ankle pronation by gently axially rotating and rolling the foot in the corrective direction by pulling from the shin assembly while further enhancing the corrective rolling with strategically placed posts on the medial edge of the sole.

US 2013/0211300 relates to a hinged ankle brace having a semi-rigid ankle cuff and a semi-rigid foot bed rotatably connected by a medial hinge and a lateral hinge stability and support to a wearer's ankle. The medial hinge of the ankle brace may be higher than the lateral hinge to accurately replicate the bending motion of an ankle.

While some of the prior art may contain some similarities relating to the present invention, none of them teach, suggested or include all of the advantages and unique features of the invention disclosed hereafter.

### SUMMARY OF THE INVENTION

The present invention comprises an ankle-foot varus deformity alignment device to improve the leg, foot and ankle alignment of a person suffering from an ankle-foot varus deformity.

The ankle-foot varus deformity alignment device comprises an elongated interconnecting member extending between an upper calf cuff and a lower foot support or platform.

The upper calf cuff comprises a collar including a medial region adapted to engage the medial side of the upper calf area to apply inward force against the calf.

The lower foot support or platform comprises a foot support plate or member including a forefoot region, a midfoot region and a hindfoot region.

A lower medial restraint extends upwardly from the medial side of the foot support plate or member shaped and dimensional to apply an inward force against the foot and ankle. A lower lateral restraint extending upwardly from the lateral side of the foot support plate or member in the hindfoot region is shaped and dimensional to apply an inward force against the lateral side of the ankle.

Thus with the counteracting forces of the upper and lower medial restraints, and the lower lateral restraint act to improve alignment of the calf, ankle and foot of a person from an ankle-font varus deformity.

The invention accordingly comprises the features of construction, combination of elements, and arrangement of parts which will be exemplified in the construction hereinafter set forth, and the scope of the invention will be indicated in the claims.

### BRIEF DESCRIPTION OF THE DRAWINGS

For a fuller understanding of the nature and object of the invention, reference should be had to the following detailed description taken in connection with the accompanying drawings in which:

FIG. 1 is a front view of the ankle-foot varus deformity alignment device of the present invention.

FIG. 2 is a medial side view of the ankle-foot varus deformity alignment device of the present invention.

FIG. 3 is a lateral side view of the ankle-foot varus deformity alignment device of the present invention.

FIG. 4 is an additional front view of the ankle-foot varus deformity alignment device of the present invention.

FIG. 5 is a cross-sectional view of the ankle-foot varus deformity alignment device of the present invention taken along line 5-5 of FIG. 4.

FIG. 6 is a cross-sectional side view of the ankle-foot varus deformity alignment device of the present invention taken along line 6-6 of FIG. 1.

FIG. 7 is a cross-sectional front view of the ankle-foot varus deformity alignment device of the present invention taken along line 7-7 of FIG. 4.

Similar reference characters refer to similar parts throughout the several views of the drawings.

### DETAILED DESCRIPTION OF THE INVENTION

The present invention comprises an ankle-foot varus deformity alignment device generally indicated as 10 to improve the leg, foot and ankle alignment of a person suffering from an ankle-foot varus deformity. The ankle-foot varus deformity alignment device 10 may be constructed of an impregnated graphite fiber material.

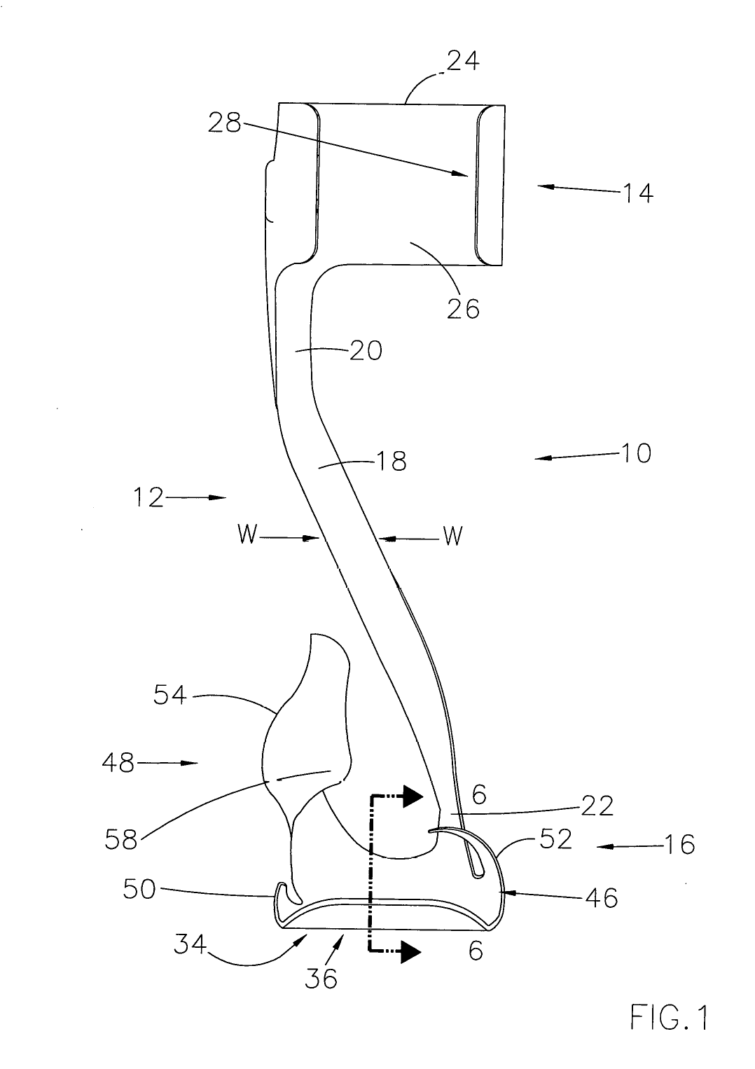

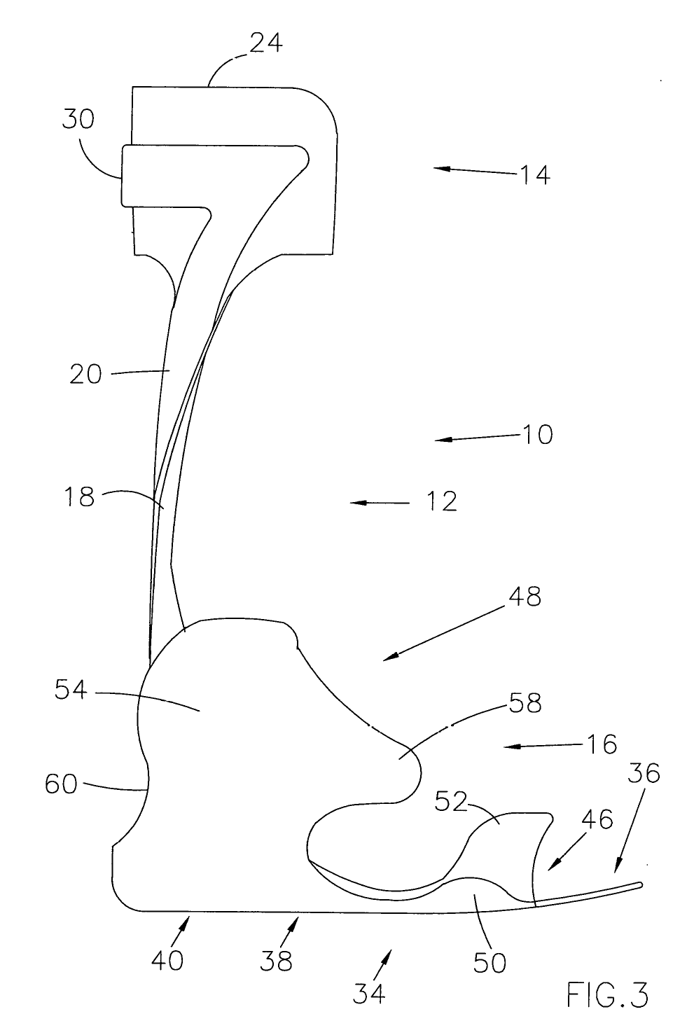

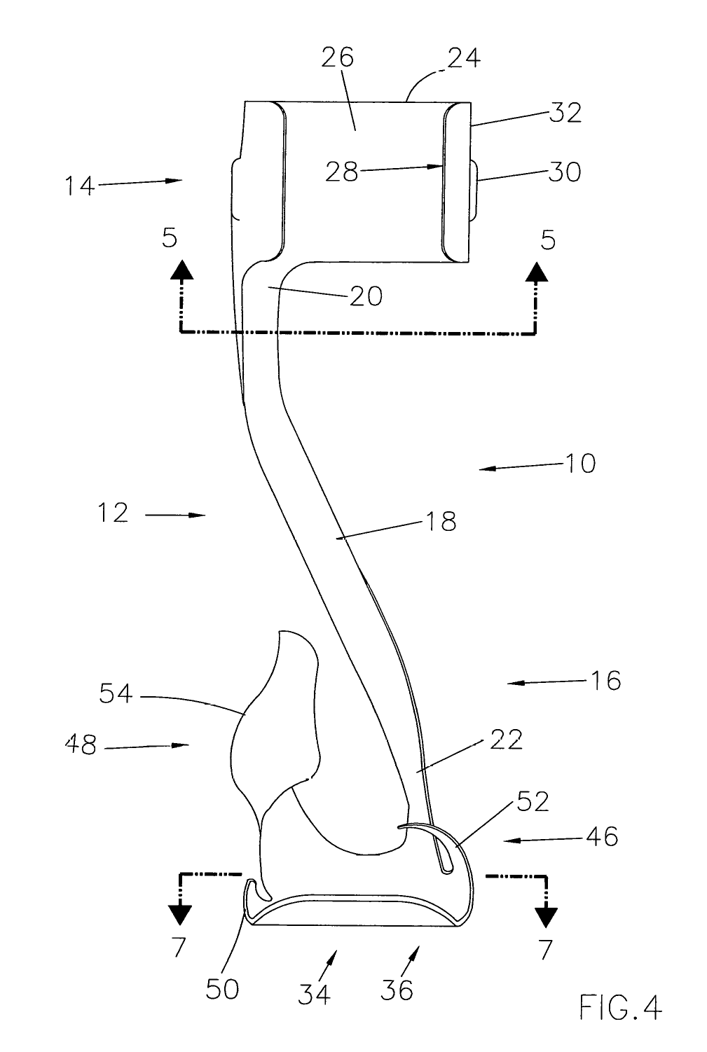

As best shown in FIGS. 1 through 4 the ankle ankle-foot varus deformity alignment device 10 comprises an elongated spiral configured interconnecting member generally indicated as 12 extending between an upper calf cuff generally indicated as 14 and a lower foot support or platform generally indicated as 16.

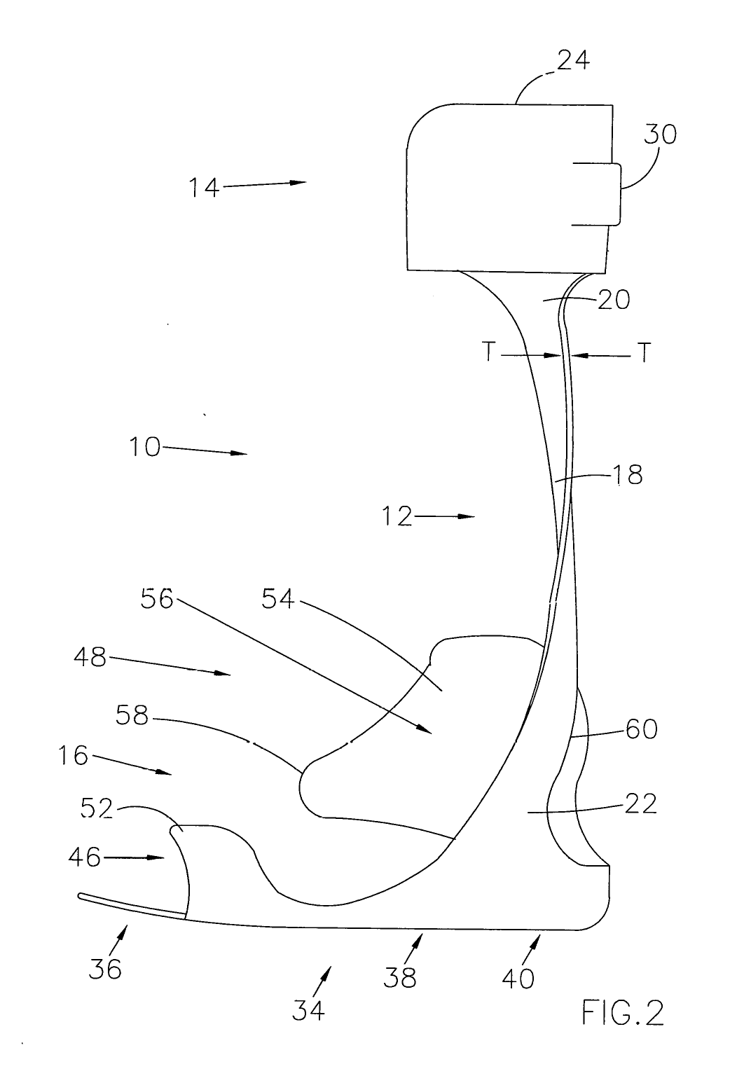

The lateral linear dimension or width W-W (FIG. 1) of the elongated spiral configured interconnecting member 12 is at least about one and one-half times the longitudinal linear dimension or thickness T-T (FIG. 2) of the elongated spiral configured interconnecting member 12. Preferably the lateral linear dimension or width W-W of the elongated spiral configured interconnecting member 12 is at least about ⅜ inch.

Since the width of the major or mid-portion 18 in the center of the elongated spiral configured interconnecting member 12 extending between the upper minor portion 20 of the elongated spiral configured interconnecting member 12 affixed to the upper calf cuff 14 and the lower minor portion 22 of the elongated spiral configured interconnecting member 12 affixed to the lower foot support or platform 16 is disposed in the plane generally perpendicular to the direction in which the wearer is walking, the elongated spiral configured interconnecting member 12 provides a limited degree of flexure to allow for a more normal walking stride.

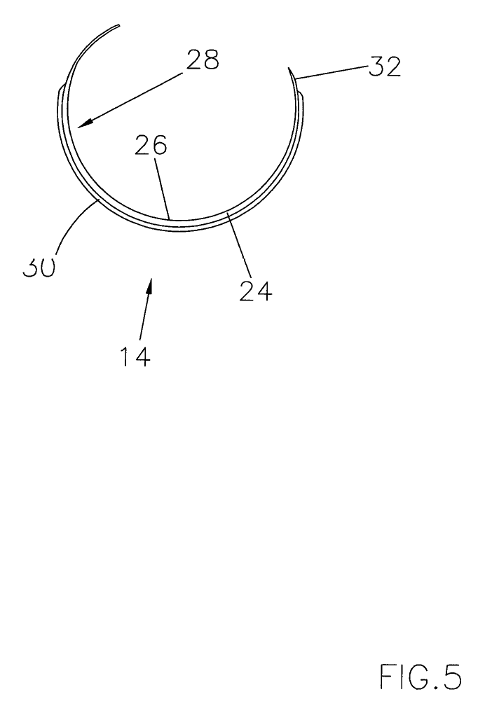

As shown in FIGS. 1 through 5, the upper calf cuff 14 comprises a substantially cylindrically shaped collar 24 including a substantially concave inner surface 26 to partially encircle the upper portion of the calf including an upper medial restraint or upper medial region generally indicated as 28 adapted to engage the medial side of the upper calf area to apply inward force against the calf. A stiffner 30 enhances the application of force against the calf about the outside surface 32 of the substantially cylindrically shaped collar 24 by reducing the flexure of the substantially cylindrically shaped collar 24.

As shown in FIGS. 1 through 4, 6 and 7, the lower foot support or platform 16 comprises a foot support plate or member generally indicated as 34 including a forefoot region generally indicated as 36, a midfoot region generally indicated as 38 and a hindfoot region generally indicated as 40.

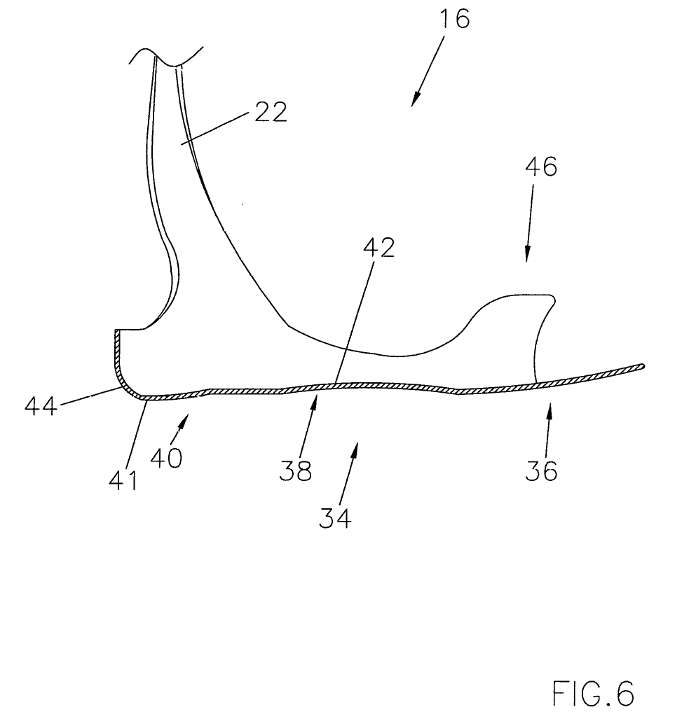

As best shown in FIG. 6, the foot support plate or member 34 is curved upwardly in the forefoot region 36 to support the toes and includes a raised portion 42 in the midfoot region 38 to support the arch. A heel cup 44 is formed in the hindfoot region 40 including a recess or depression 41 to receive and stabilize the heel.

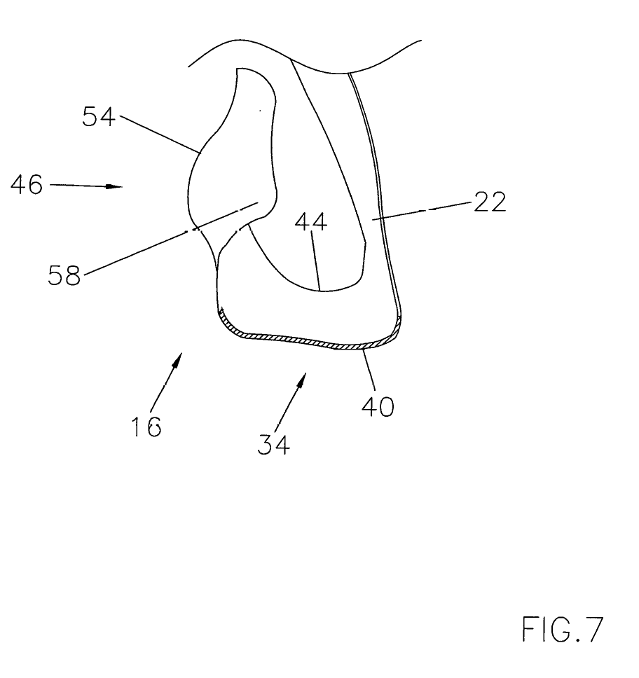

As best shown in FIG. 7, the foot support plate or member 34 in the hindfoot region 40 adjacent to the heel cup 44 is inclined or slanted downwardly from the lateral side to the medial side of the foot support plate or member 34 to roll or position the foot and ankle into alignment with the calf.

As best shown in FIGS. 1 through 4, a lower medial foot restraint generally indicated as 46 extends upwardly from the medial side of the foot support plate or member 34 generally in the area between the forefoot region 36 and the midfoot region 38 is shaped and dimensional to apply an inward force against the foot and ankle and a lower lateral ankle restraint generally indicated as 48 extends upwardly from the lateral side of the foot support plate or member 34 in the hindfoot region 40 is shaped and dimensional to apply an inward force against the lateral side of the ankle. In additional, a lateral foot restraint 50 extends upwardly from the lateral side of the foot support plate or member 34 in the midfoot region 38 to engage the outside of the foot to maintain the foot in longitudinal alignment on the foot support plate or member 34.

As best shown in FIGS. 1 through 4 and 6, the lower medial foot restraint 46 comprises an arcuate member 52 curved inwardly shaped and dimensioned to engage the inside and top of the foot in the area between the foot and toes.

As best shown in FIGS. 1 through 4 and 7, the lower lateral ankle restraint 48 comprises a lateral ankle cuff 54 including a concave recess 56 shaped and dimensioned to engage the lateral side of the ankle to restrain lateral movement thereof and force the ankle and foot inwardly or medially. Extending forwardly and rearwardly from the ankle cuff 54 is an inwardly curved instep engaging extension or restraint 58 and an inwardly curved lower leg engaging extension or restraint 60 respectively shaped and dimensioned to further restrict the lateral movement of the foot and ankle.

Thus with the counteracting forces of the upper and lower medial restraints 28 and 46 respectively, and the lower lateral restraint 48 act to align the calf, ankle and foot.

It will thus be seen that the objects set forth above, among those made apparent from the preceding description are efficiently attained and since certain changes may be made in the above construction without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Now that the invention has been described,

What is claimed is:

1. An ankle-foot varus deformity alignment device to improve leg, foot and ankle alignment of a person suffering from an ankle-foot varus deformity comprising an elongated interconnecting member extending between an upper calf cuff including a medial region adapted to engage the medial side of the upper portion of the calf and a lower foot support including a lower lateral ankle restraint adapted to engage the lateral side of the ankle and a lower medial foot restraint adapted to engage in medial side of the foot to improve leg, ankle, and foot alignment.

2. The ankle-foot varus deformity alignment device of claim 1 wherein said elongated interconnecting member comprises a spiral configuration.

3. The ankle-foot varus deformity alignment device of claim 2 wherein the lateral linear dimension or width of said elongated spiral configured interconnecting member is at least about one and one-half times the longitudinal linear dimension or thickness of said elongated spiral configured interconnecting member.

4. The ankle-foot varus deformity alignment device of claim 3 wherein the width of the major or mid-portion of said elongated spiral configured interconnecting member extending between a upper minor portion of said elongated spiral configured interconnecting member affixed to said upper calf cuff and a lower minor portion of said elongated spiral configured interconnecting member affixed to said lower foot support or platform is disposed in the plane generally perpendicular to the direction in which the wearer is walking, said elongated spiral configured interconnecting member provides a limited degree of flexure to allow for a more normal walking stride.

5. The ankle-foot varus deformity alignment device of claim 1 wherein said upper calf cuff includes a substantially concave inner surface to partially encircle the upper portion of the calf including an upper medial restraint or upper medial region adapted to engage the medial side of the upper calf area to apply inward force against the calf.

6. The ankle-foot varus deformity alignment device of claim 5 wherein said upper calf cuff further includes a stiffener to enhance the application of force against the calf by limiting the flexing of said upper calf cuff.

7. The ankle-foot varus deformity alignment device of claim 1 wherein said lower lateral ankle restraint comprises an ankle cuff including a concave recess shaped and dimensioned to engage the lateral side of the ankle to restrain lateral movement thereof and force the ankle and foot inwardly or medially.

8. The ankle-foot varus deformity alignment device of claim 7 further including an inwardly curved instep engaging extension or restraint extending forwardly and rearwardly from said ankle cuff and an inwardly curved lower leg engaging extension or restraint shaped and dimensional to further restrict the lateral movement of the foot and ankle.

9. The ankle-foot varus deformity alignment device of claim 1 wherein said lower foot support or platform comprises a foot support plate or member including a forefoot region, a midfoot region and a hindfoot region.

10. The ankle-foot varus deformity alignment device of claim 9 wherein said lower medial foot restraint extends upwardly from the medial side of said foot support plate or member between said forefoot region and said midfoot region shaped and dimensioned to apply an inward force against the foot and ankle.

11. The ankle-foot varus deformity alignment device of claim 10 further including a comprising lower medial foot restraint comprising an arcuate member curved inwardly shaped and dimensioned to apply an inward force against the inside and top of the foot in the area between the foot and toes.

12. The ankle-foot varus deformity alignment device of claim 9 wherein said lower lateral ankle restraint extends upwardly from the lateral side of said foot support plate or member in said hindfoot region shaped and dimensioned to apply an inward force against the lateral side of the ankle.

13. The ankle-foot varus deformity alignment device of claim 9 wherein said lower lateral ankle restraint comprises an ankle cuff including a concave recess shaped and dimensioned to engage the lateral side of the ankle to restrain lateral movement thereof and force the ankle and foot inwardly or medially.

14. The ankle-foot varus deformity alignment device of claim 13 further including an inwardly curved instep engaging extension or restraint extending forwardly and rearwardly from said ankle cuff and an inwardly curved lower leg engaging extension or restraint shaped and dimensioned to further restrict the lateral movement of the foot and ankle.

15. The ankle-foot varus deformity alignment device of claim 9 further including a lateral foot restraint extending upwardly from the lateral side of the foot support plate or member in the midfoot region to engage the outside of the foot to maintain the foot in longitudinal alignment on said foot support plate or member.

16. The ankle-foot varus deformity alignment device of claim 9 wherein said foot support plate or member in said hindfoot region is inclined or slanted downwardly from the lateral side to the medial side of said foot support plate or member to roll or force the foot and ankle into alignment with the calf.

17. The ankle-foot varus deformity alignment device of claim 9 further including a heel cup formed in said hindfoot region including a depression to receive and stabilize the heel.

18. The ankle-foot varus deformity alignment device of claim 9 wherein said lower medial foot restraint extends upwardly from the medial side of said foot support plate or member between said forefoot region and said midfoot region shaped and dimensioned to apply an inward force against the foot and ankle and further including a lower medial foot restraint comprising an arcuate member curved inwardly shaped and dimensioned to apply an inward force against the inside and top of the foot in the area between the foot and toes and said lower lateral ankle restraint extends upwardly from the lateral side of said foot support plate or member in said hindfoot region shaped and dimensioned to apply an inward force against the lateral side of the ankle.

19. The ankle-foot varus deformity alignment device of claim 18 further including said lower lateral ankle restraint comprises an ankle cuff including a concave recess shaped and dimensioned to engage the lateral side of the ankle to restrain lateral movement thereof and force the ankle and foot inwardly or medially.

20. The ankle-foot varus deformity alignment device of claim 19 further including an inwardly curved instep engaging extension or restraint extending forwardly and rearwardly from said ankle cuff and an inwardly curved lower leg engaging extension or restraint shaped and dimensioned to further restrict the lateral movement of the foot and ankle.

21. The ankle-foot varus deformity alignment device of claim 20 further including a lateral foot restraint extending upwardly from the lateral side of the foot support plate or member in the mid-foot region to engage the outside of the foot to maintain the foot in longitudinal alignment on said foot support plate or member.

22. The ankle-foot varus deformity alignment device of claim 20 wherein said foot support plate or member in said hindfoot region is inclined or slanted downwardly from the lateral side to the medial side of said foot support or plate or member to roll or position the foot and ankle into alignment with the calf.

23. The ankle-foot varus deformity alignment device of claim 20 further including a heel cup formed in said hindfoot region including a depression to receive and stabilize the heel.

24. The ankle-foot varus deformity alignment device of claim 9 wherein said lower medial foot restraint extends upwardly from the medial side of said foot support plate or member between said forefoot region and said mid-foot region shaped and dimensioned to apply an inward force against the foot and ankle and said lower lateral ankle restraint extends upwardly from the lateral side of said foot support plate or member in said hindfoot region shaped and dimensioned to apply an inward force against the lateral side of the ankle.

25. The ankle-foot varus deformity alignment device of claim 24 further including a lower medial foot restraint comprising an arcuate member curved inwardly shaped and dimensioned to apply an inward force against the inside and top of the foot in the area between the foot and toes and said lower lateral ankle restraint comprises an ankle cuff including a concave recess shaped and dimensioned to engage the lateral side of the ankle to restrain lateral movement thereof and force the ankle and foot inwardly or medially.

26. The ankle-foot varus deformity alignment device of claim 25 further including a lateral foot restraint extending upwardly from the lateral side of the foot support plate or member in the mid-foot region to engage the outside of the foot to maintain the foot in longitudinal alignment on said foot support plate or member.

27. The ankle-foot varus deformity alignment device of claim 26 wherein said foot support plate or member in said hindfoot region is inclined or slanted downwardly from the lateral side to the medial side of said foot support plate or member to roll or force the foot and ankle into alignment with the calf.

28. The ankle-foot varus deformity alignment device of claim 24 further including an inwardly curved instep engaging extension or restraint extending forwardly and rearwardly from said ankle cuff and an inwardly curved lower leg engaging extension or restraint shaped and dimensioned to further restrict the lateral movement of the foot and ankle.

29. The ankle-foot varus deformity alignment device of claim 28 further including a lateral foot restraint extending upwardly from the lateral side of the foot support plate or member in the midfoot region to engage the outside of the foot to maintain the foot in longitudinal alignment on said foot support plate or member.

30. The ankle-foot varus deformity alignment device of claim 29 wherein said foot support plate or member in said hindfoot region is inclined or slanted downwardly from the lateral side to the medial side of said foot support plate or member to roll or force the foot and ankle into alignment with the calf.
